(19) United States
(12) Patent Application Publication
(10) Pub. No.: US 2004/0043427 A1
(43) Pub. Date: Mar. 4, 2004

(54) Molecular bioswitch for detecting protein interactions using electrical conductivity

(76) Inventor: Stewart J. Lebrun, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/608,903

(22) Filed: Jun. 27, 2003

Related U.S. Application Data
(60) Provisional application No. 60/393,195, filed on Jul. 1, 2002.

(51) Int. Cl. G01N 33/53

(52) U.S. Cl. 435/7.1

(57) ABSTRACT

Preferred aspects of the present invention relate to a method for detection and quantification of molecular interactions between proteins bound to a support and ligand molecules, in particular antibodies, in a test sample. More particularly, the preferred methods takes advantage of the electrical conductivity of metal-antibody conjugates.

### BACKGROUND OF THE INVENTION

1. Field of the Invention

Preferred aspects of the present invention relate to a rapid method for detection and quantification of molecular interactions between proteins bound to a support and ligand molecules, in particular antibodies, in a test sample. More particularly, the preferred methods takes advantage of the electrical conductivity of metal-antibody conjugates.

2. Description of the Related Art

Protein arrays have been used to facilitate rapid screening of protein-ligand interactions. Proteomics or the large scale understanding of proteins is still in its infancy and is becoming increasingly important. Many new tools and techniques need to be developed in order to study the function of all the proteins of a cell, tissue, and eventually organism in the same efficient manner as that of DNA microarrays. Protein arrays are described, for example, in PCT/US00/06244, which is incorporated herein in its entirety by reference thereto. The high through-put potential of protein arrays, however, is limited by the availability of high-sensitivity analytical methods for detection of protein interactions.

Prior art methods for high-sensitivity detection of chemical and biological materials have typically relied on colorimetric analyses of enzymatic or immunological reactions of the test analytes. Enzyme immunoassay (EIA) techniques have been incorporated into a large number of biosensor devices employing calorimetric or fluorimetric analyses, as exemplified in the following U.S. and foreign patents: U.S. Pat. No. 4,343,782; EP 125,554; EP 128,318; EP 231,010; EP 288,256; EP 290,269; SU 1,189,224; JP 57,208,457. In a typical embodiment, an enzyme-conjugated antigen admixed with immobilized antibody reacts competitively with free test antigen to release the enzyme into solution containing its substrate, and subsequent enzyme reaction products are detected by changes in color or fluorescence. A drawback of this method is background color which limits sensitivity.

Biosensors incorporating electrochemical detection with adsorbed, deposited, or embedded receptor agents on an electrode are exemplified in the following U.S. and foreign patents: U.S. Pat. No. 4,634,599; U.S. Pat. No. 4,661,442; JP 63,153,462; JP 63,206,652; JP 01,59,058; EP 304,447; WO 87,03,095; WO 88,08,972; WO 88,09,499; and WO 88,09,808. In these systems, analyte detection is typically followed by a change in electrical potential or current at an electrode surface. Sensitivity and selectivity for specific analytes depend on the nature of the materials used on the electrode, and have limited the applicability of these devices.

Japanese Patent No. 63,153,462 to Takei et al., published Jun. 25, 1988, describes a sensitive field effect transistor biosensor containing an aluminum gate-immobilized biological substance. European Patent No. 304,947 to Kagayama, published Mar. 1, 1989, describes a biosensor containing immobilized physiologically active substance and transducer, comprising a pair of opposing flat plates between which an antibody, with antigen or enzymes immobilized onto one surface and a transducing chemical substance on the other surface.

PCT International Application No. WO 88,08,972 to Cheung et al., published Nov. 17, 1988, describes a biosensor comprising a reversibly selective binding protein immobilized upon the insulated gate region of a field effect transistor on the sensor. PCT International Application No. WO 88,09,808 to Taylor and Marenchic, published Dec. 15, 1988, describes receptor-based biosensors and a method of immobilizing and stabilizing an active biological receptor in a polymeric film onto an electrode. PCT International Application No. WO 88,09,499 to Newman, published Dec. 1, 1988, describes an optimized capacitive sensor for chemical analysis, which relies on biospecific binding between a biochemical binding system and the analyte of interest to change the dielectric properties of a capacitive affinity sensor.

Japanese Patent No. 01,59,058 to Kuriyama, published Mar. 6, 1989, describes an enzyme immunosensor and its use in enzyme immunoassay, consisting of a semiconductor ion sensor, a spacer, and plate containing immobilized antigen or antibody. PCT International Application No. WO 87,03,095 to Newman, published May 21, 1987, describes a capacitive affinity sensor and method for chemical analysis and measurement, comprising an open capacitor which produces a higher electric field in one volumetric region and a lower field in a second region, a biospecific binding agent for the analyte localized on the surface between the conductors in the first region, and a means associated with the capacitor which responds to the average dielectric constant in the first chamber.

A biosensor for detection of hazardous chemicals or toxins incorporates is disclosed in U.S. Pat. No. 5,328,847, which is incorporated herein in its entirety by reference thereto. In this patent, a modular electrochemical biosensor for chemical and biological target agents is disclosed, which incorporates a reaction between the target agent of interest and a recognition biomolecule in a biochemical switch module coupled with a gated membrane electrode in an indicator module.

None of the prior art biosensors provide a simple conductivity-based molecular bioswitch that can be adapted for array-based analysis of protein-antibody interactions using electrochemical detection, wherein the same detection chemistry can be applied regardless of the nature of the protein antigens on the array.

### SUMMARY OF THE INVENTION

### BRIEF DESCRIPTION OF THE DRAWINGS

FIG. 1 is a schematic representation of a system in accordance with one embodiment of the present invention.

FIG. 2 is a plot of the capacitance (nF) of nickel anti-human IgG (AP) conjugates as a function of the amount of antibody ranging from 0 μl/ml to 100 μl/ml for four different percentages of nickel (0%, 0.1%, 0.5%, and 1%). This experiment was conducted at room temperature (˜25° C.) and room humidity (˜50% to 60%).

FIG. 3 is a plot of the current (μA) of nickel anti-human IgG (AP) conjugates as a function of the amount of antibody ranging from 0 μl/ml to 100 μl/ml for four different percentages of nickel (0%, 0.1%, 0.5%, and 1%).

FIG. 4 is a plot of the capacitance (nF) of nickel anti-human IgG (AP) conjugates as a function of the amount of antibody ranging from 0 μl/ml to 20 μl/ml for four different percentages of nickel (0%, 0.1%, 0.5%, and 1%). The plotted data combines three different trial runs, and isolates the amount of antibody to 0 μl/ml to 20 μl/ml. This is the antibody range that peaked in FIG. 2.

FIG. 5 is a plot of the current (μA) of nickel anti-human IgG (AP) conjugates as a function of the amount of antibody ranging from 0 μl/ml to 20 μl/ml for four different percentages of nickel (0%, 0.1%, 0.5%, and 1%). The plotted data combines three different trial runs, and isolates the amount of antibody to 0 μl/ml to 20 μl/ml. This is the antibody range that peaked in FIG. 3.

FIG. 6 is a plot of the capacitance change of N2 and C2 streaked chips in a dry environment over a period of 26 days.

### DETAILED DESCRIPTION OF THE PREFERRED EMBODIMENT

In one preferred embodiment, the present invention is related to a rapid and economical method for detecting and quantifying molecular interactions between proteins and antibodies. Briefly, antibodies are labeled with some conductive element (such as Mg, Mn, Fe, Co, Ni, Ga, Pd, Ag, or Cd; collectively referred to hereinafter as a metal, “X”) via oxidation of tyrosyl or histidyl side chains, where some metal is titrated with the monoclonal or polyclonal antibody (hereinafter “ab”) to be labeled. The metal-antibody conjugates (hereinafter “X-ab”) are preferably formed by enzymatic or chemical oxidation, wherein reaction time can easily be optimized. For example, labeling can be induced using the Bolter-Hunter reaction, where N-succinimidyl-3-(hydroxyphenyl) propionate (Bolton-Hunter reagent) couples free amino groups on amino-terminal residues with the metal ion. Alternatively, the antibody can be dialyzed in doubled-distilled H₂O, wherein the X-ab conjugation reaction occurs in ion free H₂O. This is accomplished by reacting a solution of XCl or XCl₂ with buffer free ab. The reaction is preferably carried out at 0-20° C., more preferably at about 4° C., for a period of between about 12-72 hrs, preferably about 48 hr. The metal, which is the only oxidizing agent in solution, attaches to tyrosine side chains, thereby forming the X-ab conjugate.

The formation of X-ab follows the reaction: X+ab [symbol] X-ab+X+ab. The X-ab conjugate is separated from free X and ab using standard purification techniques. For example, initial purification can be accomplished by molecular exclusion chromotagraphy using a gel matrix with an exclusion limit of about 20,000 to 50,000 daltons. The metal-labeled antibody can then be dialyzed against 2× 1-L buffer to remove any unincorporated metal (for example, using KPO₄ buffer at pH 7.4, containing glycerol, NaCl, and DTT). Final purification preferably involves adding a magnetic coil to a buffer solution and inducing a magnetic field to sequester the X-ab conjugates. The coil with voltage is then transferred to fresh buffer, allowing the X-ab conjugates to diffuse into solution once the voltage is turned off. Preferably, the solution is allowed to sit at 4° C. overnight. This final purification is not essential as the conductance of the X-ab conjugates is being measured, wherein only the metal conducts and any unlabeled ab will not contribute to the conductance.

In a preferred embodiment, the X-ab conjugate is tested by streaking it onto a solid substrate, preferably a hydrophobic substrate such as a PDVF membrane, and more preferably a ζ-Grip™ microarray chip (Miragene, Inc., Irvine, Calif.). After the X-ab streak has dried, conductance is measured using a voltmeter by applying a voltage across the streak. If the metal complex is present on the substrate a current will be conducted across the streak.

In preferred embodiments, the ab portion of the X-ab conjugate exhibits specific binding to proteins laid down on the substrate. In contrast, the X portion of the X-ab conjugate preferably exhibits no specificity toward the substrate or proteins.

In accordance with one preferred aspect of the present biomolecular switch, the above-described X-ab and substrate can be used to distinguish diseased serum (comprising for example, the patient's antibodies against a disease pathogen, or a patient's autoantibodies in an autoimmune disorder) from healthy, non-immune serum (comprising for example, no specific antibodies against the disease pathogen or autoimmune antigen, etc.). The procedure involves spotting an antigen (e.g., a pathogenic antigen or an autoimmune antigen) onto a substrate, in accordance with known methods for preparing a protein microarray (see e.g., co-pending U.S. patent application Ser. No. 10/376,351; incorporated in its entirety herein by reference thereto). In preferred embodiments of the present invention, the antigen is spotted onto a ζ-Grip™ microarray chip. After blocking to minimize non-specific binding, the substrate is incubated with the test serum. Preferably, a parallel substrate, spotted with the antigen is blocked and incubated with a control (healthy) serum sample. After washing, the substrate containing any antigen-antibody complexes, is incubated with the X-ab solution, wherein the ab is specific for the patients antibodies (for example, an X-labeled anti-human IgG). The addressable antigen spots are analyzed for the presence of metal (ability to conduct an electric current) by passing a current across the spot between electrodes and measuring electrical conductivity, preferably using a digital multimeter.

In accordance with another preferred aspect of the present biomolecular switch, the above-described X-ab and substrate can be used as a highly sensitive digital detection means for detecting the presence of an analyte in a sample. In this variation, unlabeled antibodies (“ab₁”), which are specific to an epitope (“Ep₁”) of a particular antigen (“Ag,” e.g., anthrax protective antigen), are laid down on a substrate, e.g., preferably a streak on ζ-Grip™. After blocking, an unknown sample comprising a mixture of possible antigens being screened for the present of Ag is then contacted with the substrate comprising unlabeled ab₁. X-ab conjugate is then added. In one embodiment the ab portion of the X-ab conjugate is ab₁, which is specific for the same epitope Ep₁ on the Ag. In a variation, the ab portion of the X-ab conjugate is ab₂, which is specific for a second epitope Ep₂ on the Ag. The streak (or spot) is then analyzed for the presence of metal (ability to conduct an electric current) by passing a current across the streak (or spot) between electrodes and measuring electrical conductivity, preferably using a digital multimeter. Using a test cassette configured to accept the substrate slide between built-in electrodes, connected to a digital multimeter, which is connected to a data analysis/storage module, will allow nearly instantaneous testing for the presence of a particular antigen of interest.

In another embodiment, protein degradation can be monitored by the above-described system. Applicant has found that the level of specific X-ab binding to protein antigens decreases with time as a function of the extent of protein degradation. Thus, protein integrity can be readily tested in accordance with an embodiment of the present invention by laying down a solution containing the test protein in a streak or spot on a substrate, preferably ζ-Grip™, and then probing for epitope integrity by assessing the level of X-ab binding—by measuring conductance as detailed above, and in the specific examples below. Resistance will increase (conductivity will decrease) with degradation of the protein.

In another embodiment, the above-described system can be modeled as a capacitor in parallel with a resistor.

It should be understood that although X-ab is used to provide the following proof-of-concept, any other protein can be used in place of the ab. For example, receptor-ligand binding can be exploited in the same manner as discussed above for antibody-antigen interactions. Likewise, enzyme-substrate pairs can also be used. Indeed, any protein-ligand pair can be utilized, wherein one of the binding pair is labeled with the metal, and the other is immobilized on a substrate, either directly, or indirectly, for example in a sandwich-type assay.

It should also be clear to the skilled artisan that fragments of the protein or ligand, e.g., binding epitopes, active sites, antigenic determinants, etc. can also be employed in aspects of the present invention.

With regard to detecting the capacity of a “developed” spot or streak (e.g., contacted with the X-ligand conjugate) to conduct an electric current, it should be noted that the electrode placement and design can be configured by the skilled artisan to allow detection of an electric voltage or current across the developed spot or streak. For example, the substrate could be formed on an integrated circuit chip wherein microelectrodes surround each spot in a microarray, as will be known in the art. Alternatively, a microelectrode array could be placed in contact with the substrate, wherein a plurality of electrode pairs are positioned relative to the addressable spots so that conductivity can be assessed between the electrodes and across the spot.

The protein binding portion of the chip may be coated with any polymeric substrate adapted to immobilize a protein. PVDF and nitrocellulose are prefered. Examples of suitable polymers include a polyamide, polysulfone, polyolefin, polyhalogenatedolefin, polystyrene, polyol, polvamine, polyimine, polyester, anacrylic polymer, polyacrylic acid, polyacrylic ester, polyhydroxyalkyl acrylate, polyacrylic amide, polyacrylonitrile, polyvinyl heterocyclic, polyheterocyclic, polycarbonate,polyimide, polyamide-imide, polylactide, polyglycolide, polyglycolide/lactide, polypeptide, polysiloxane, polysilane, polyacetylene, polyphosphazene, polysaccharide, polyether, epoxy resin, polyacetal, polyurethane, polyurea, urea-formaldehyde resin, polyphenol, phenol-formaldehyde resin, alkyd resin, melamine-formaldehyde resin, a dendrimer, a spiro polymer, polyaryleneoxide, polysulfide, polyketone, polyetherketone, polyetheretherketone, polyaromatic, polyaldehyde. allyl resin, cellulose, cellulose ester, cellulose derivative, and combinations thereof.

An example of a polyhalogenated olefin is polyvinylidene fluoride (PVDF). For example, blends of two or more of the above polymers can be employed, and copolymers comprising monomer segments of one or more of these polymers can be employed. Examples of cellulose derivatives include ethyl cellulose, hydroxyethyl cellulose and hydroxypropyl cellulose. Examples of cellulose esters include the nitrates., acetates, propionates, and butyrates of cellulose. Examples of charged polymers include a polvamide, polyamine, polyimine, polyacrylic amide, polyvinyl heterocyclic, polyheterocyclic, polyimide, polvamideimide, polypeptide, polyurethane, polyurea. urea-formaldehyde resin, melamine formaldehyde resin, a dendrimer, and cellulose derivatives. Examples of hydrophilic polymers include a polvamide, polyol, polyamine, polvimine, polyester, an acrylic polymer, polyacrylic acid, polyacrylic ester, polyhydroxyalkyl acrylate, polyacrylic amide, polyacrylic nitrile, polyvinyl heterocyclic, polyheterocyclic, polyimide, polyamide-imide, polylactide, polypeptide, polysaccharide, polvether, epoxy resin, polyacetal, polyurethane, polyurea, urea-formaldehyde resin, polyphenol, phenol-formaldehyde resin, alkyd resin, melamine-formaldehyde resin, a dendrimer, a spiro polymer, polysulfide, polyketone, polvaldehyde, cellulose, cellulose ester or cellulose derivative; a polysulfone, polyolefin, polyhalogenated olefin, polystyrene polycarbonate, polysiloxane, polysilane, polyacetylene, polyphosphazene, polvaromatic, polyaryleneoxide, allylresin, polyetheretherketone, andpolvetherketone, and combinations thereof.

Particular examples of polar polymers include a polyamide, polysulfone, polyol, polyamine, polyimine, polyester, an acrylic polymer, polyacrylic acid, polyacrylic ester, polyhydroxyalkyl acrylate, polyacrylic amide, polyacrylic nitrile, polyvinyl heterocyclic, polyheterocyclic, polycarbonate, polyimide, polyamide-imide, polylactide, polypeptide, polysaccharide, polyether, epoxy resin, polyacetal, polyurethane, polyurea, ureaformaldehyde resin, polyphenol, phenol-formaldehyde resin, alkyd resin, melamineformaldehyde resin, a dendrimer, a spiro polymer, polyaryleneoxide, polysulfide, polyketone, polyetherketone, polyetheretherketone, polyaromatic, polyaldehyde, cellulose, cellulose ester, or cellulose derivative; a polyolefin, polyhalogenated olefin, polysiloxane, polyacetylene, polyphosphazene, polystyrene, polysilane, and combinations thereof.

Preferably, the polymer layer includes a polyamide, copolyamide, polysulfone, or polvvinylidene fluoride. Particular examples of polyamides and copolyamides include nylons, e.g., nylon 4, nylon 45, nylon 6, nylon 66, nylon 11, nylon 610, nylon 612, and nylon 6T. Nylon 66 is a further preferred polyamide.

### WORKING EXAMPLES

Preparation of metal-antibody (X-ab) conjugates—The following metal salts were used: Cadmium Chloride Anhydrous (hereafter referred to as CdCl₂), Sigma; Cobalt Chloride Hexahydrate (hereafter referred to as CoCl₂), Sigma: Cupric Sulfate Pentahydrate (hereafter referred to as CuSO₄.5H₂O), Sigma; Nickel Chloride Hexahydrate (hereafter referred to as NiCl₂.6H₂O), Sigma; and Iron(II) Sulfate Heptahydrate (hereafter referred to as FeSO₄), Aldrich. The antibody used was an anti-human IgG antibody labeled with alkaline phosphatase (hereafter referred to as γ-Human IgG-AP), Pierce Chemical. For the dialysis of X-ab, 10×PBS without calcium or magnesium, BioWittaker (cat# 03367), and enzyme grade glycerol, FisherBiotech (cat # BP229-1) were utilized. The substrate used in these examples was ζ-Grip™ microarray chips (Miragene, Inc., Irvine, Calif.). To measure the conductance and current, a multimeter (BK Tool Kit 2707A), BK Precision was used in connection with a power supply (MW122A multi-voltage 2 Amp DC Power Supply).

To test for diseased serum, SSA/Ro antigen, Immunovision, was used along with Casein in TBS Blocker, Pierce Chemical. SLE patient serum (which has previously been tested positive for the SSA/Ro antigen) and control serum, pro-medDX, was also used. For parallel colorimetric quantification of the antigen-ab interaction, the BNCIP reagent (Pierce Chemical) was used.

To create the metal antibody conjugate, 0.05 g of metal salt was dissolved in 50 ml of sterile ddH₂O. Five metals were tested—CdCl₂, CoCl₂, CuSO₄.5H₂O, NiCl₂.6H₂O, and Iron(II) Sulfate Heptahydrate FeSO₄. Antibody (100 μl of γ-Human IgG-AP) was added to 1 ml of the dissolved metal solution (1 mg/50 ml) and stored on ice, such that the following reaction occurred: X+ab [symbol] X-ab+X+ab.

After incubating at 4° C. overnight, Ni-ab and C-ab (where C defines a control serum) against 1-L of 1×PBS/10% glycerol (hereafter referred to as Buffer A) is dialyzed. Buffer A was made by mixing 100-ml of 10× PBS, 100-ml of glycerol, and ddH₂O up to 1000-ml in a 1000-ml bottle. A (cellulose) dialysis membrane was stirred in this buffer at 4° C., where the dialysis membrane contained either Ni-ab or C-ab in Buffer A (Ni-ab and C-ab containing dialysis membranes were stirred in separate beakers). This process allows for smaller molecules (in this case, free metal) to elute out of the dialysis membrane, and for the larger molecules (i.e., the antibody and metal-antibody complex) to remain in the membrane. Every 2-hrs for 6-hrs, the 1-L of Buffer A was changed. The solution in the membrane was then transferred to individual test tubes for storage.

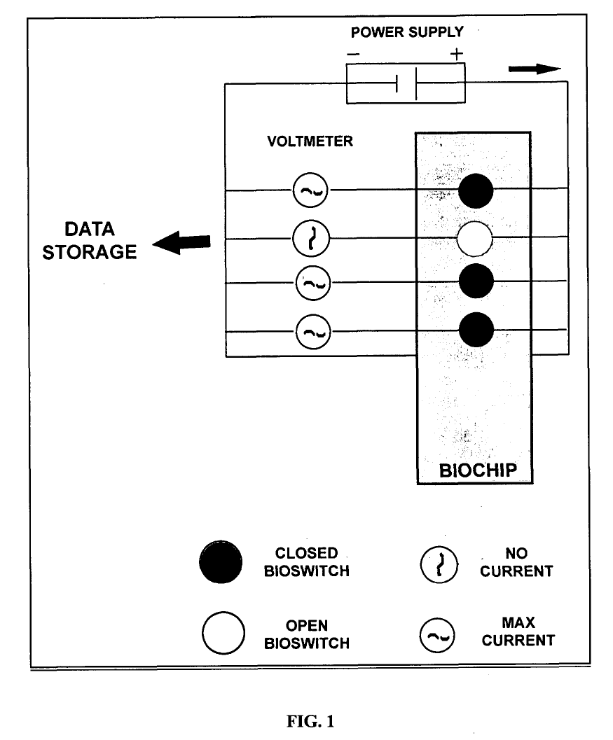

About 75-μl of the dialysed X-ab solution is streaked onto the ζ-Grip™ microarray chips, where the solution is then allowed to dry. Banana clips (attached to some power supply and voltmeter) are attached to each end of the streak on the chip. Applying a 6 V voltage from the power supply to the chip to the multimeter allows for the conductance and current across the chip to be measured. A schematic of this set up is shown in FIG. 1. If the metal complex is present on the chip, the “circuit” closes, producing a current, which is representive of the X-ab conductance on the chip.

In order to test for diseased serum, a line of the SSA/Ro antigen was run across the ζ-Grip™ microarray chip, the location marked (with a permanent marker), and the serum allowed to dry overnight. Then, the chips underwent a standard autoantigen assay: Once dried, the chips were incubated in blocker for 1-hr with agitation, followed by three washes in 1×PBS buffer (10-min each wash). About 100-μl of serum and 10-ml of 1×PBS is then used to incubate the chip for 1-hr with agitation. The chips are then washed three times in 1×PBS buffer (10-min each wash). Following, the Ni-ab solution is added to 10-ml of 1×PBS, so that the chips can then incubated in this solution for 1-hr with agitation. After washing three times with PBS, the chips are dried and the capacitance and current measured as described above. Colorimetrically, the line can be developed by incubating the chip in 10-ml of BNCIP reagent for 15-min, and stopping the reaction with ddH₂O.

### EXPERIMENTAL RESULTS

FIG. 1 is a schematic representation of a system in accordance with one embodiment of the present invention. By applying a voltage across a chip, conductance of a metal antibody complex can be measured. If the metal complex is present on the chip, the “circuit” closes, producing a current, which is representative of the X-ab conductance on the chip.

The acquired data of duplicates of three different streaking condition on the ζ-Grip™ microarray chips. NiCl₂-1 and NiCl₂-2 are two different chips, both streaked with NiCl₂ solution are shown in TABLE 1. C2-1 and C2-2 are two different chips, both streaked with anti-human IgG (AP) antibody solution only. N2-1 and N2-2 are two different chips, both streaked with the Nickel-anti-human IgG (AP) complex solution. Once dried, results were recorded in terms of each chips' current (μA), resistance (MΩ), and capacitance (nF).

TABLE 1

| | Current (μA) | Resistance (MΩ) | Capacitance (nF) |
|---|---|---|---|
| NiCl₂-1 | 0 | >200 | 0 |
| | 0 | >200 | 0 |
| | 0 | >200 | 0 |
| NiCl₂-2 | 0 | >200 | 0 |
| | 0 | >200 | 0 |
| | 0 | >200 | 0 |
| C2-1 | 0.7 | 23 | 10.6 |
| | 0.6 | 20 | 10.6 |
| | 0.6 | 20 | 10.6 |
| C2-2 | 1.3 | 12.5 | 10 |
| | 1.2 | 16 | 10 |
| | 1.2 | 16 | 10 |
| N2-1 | 5.9 | 3 | 140 |
| | 6.0 | 3 | 140 |
| | 6.0 | 2.6 | 140 |
| N2-2 | 4.6 | 3.2 | 85 |
| | 4.7 | 2.8 | 85 |
| | 4.7 | 2.9 | 85 |

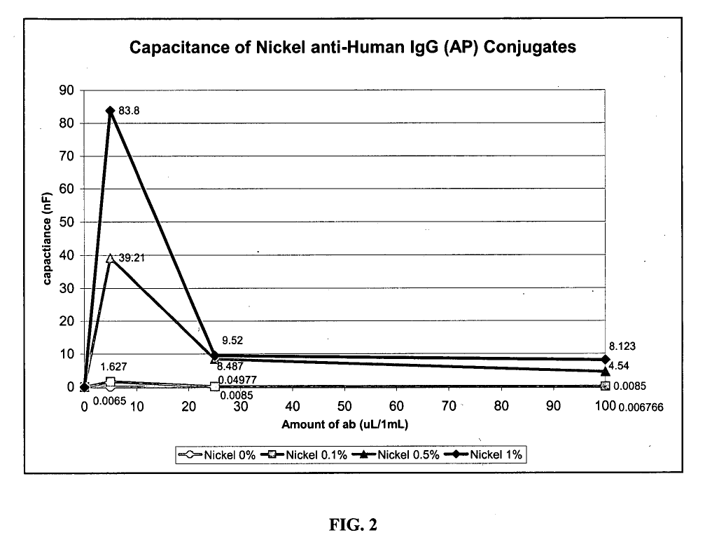

FIG. 2 is a plot of the capacitance (nF) of nickel anti-human IgG (AP) conjugates as a function of the amount of antibody ranging from 0 μl/ml to 100 μl/ml for four different percentages of nickel (0%, 0.1%, 0.5%, and 1%). This experiment was conducted at room temperature (˜25° C.) and room humidity (˜50% to 60%).

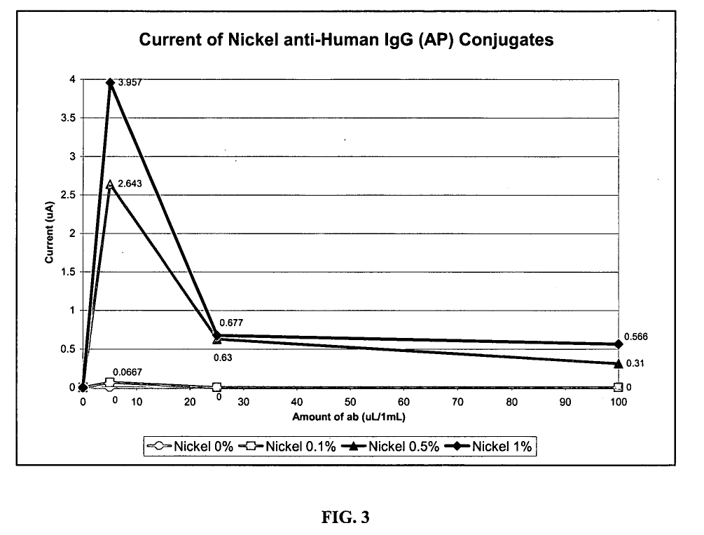

FIG. 3 is a plot of the current (μA) of nickel anti-human IgG (AP) conjugates as a function of the amount of antibody ranging from 0 μl/ml to 100 μl/ml for four different percentages of nickel (0%, 0.1%, 0.5%, and 1%). This experiment was conducted at room temperature (˜25° C.) and room humidity (˜50% to 60%).

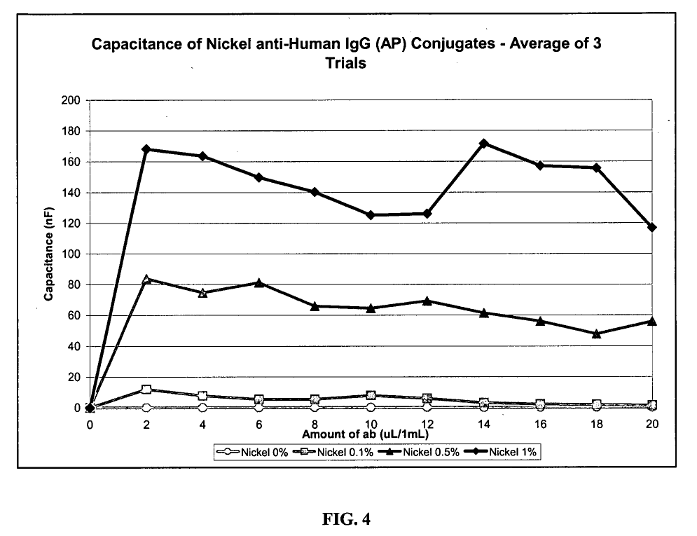

FIG. 4 is a plot of the capacitance (nF) of nickel anti-human IgG (AP) conjugates as a function of the amount of antibody ranging from 0 μl/ml to 20 μl/ml for four different percentages of nickel (0%, 0.1%, 0.5%, and 1%). The plotted data combines three different trial runs, and isolates the amount of antibody to 0 μl/ml to 20 μl/ml. This is the antibody range that peaked in FIG. 3. Again, this experiment was conducted at room temperature and room humidity.

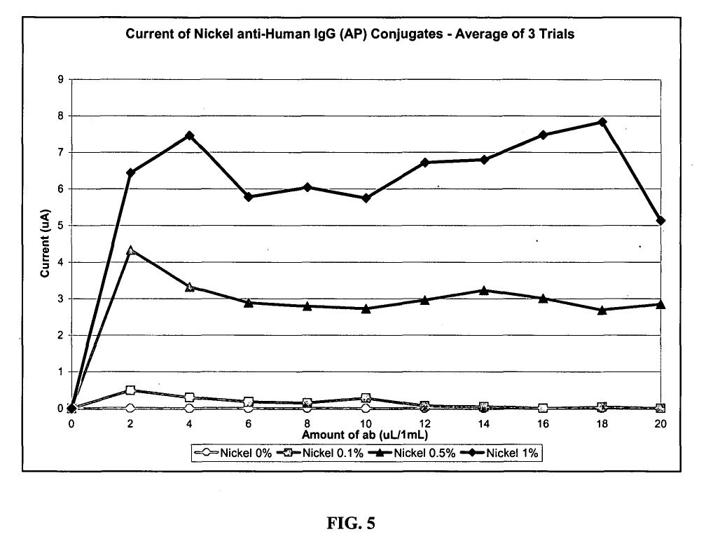

FIG. 5 is a plot of the current (μA) of nickel anti-human IgG (AP) conjugates as a function of the amount of antibody ranging from 0 μl/ml to 20 μl/ml for four different percentages of nickel (0%, 0.1%, 0.5%, and 1%). The plotted data combines three different trial runs, and isolates the amount of antibody to 0 μl/ml to 20 μl/ml. This is the antibody range that peaked in FIG. 4. Again, this experiment was conducted at room temperature and room humidity.

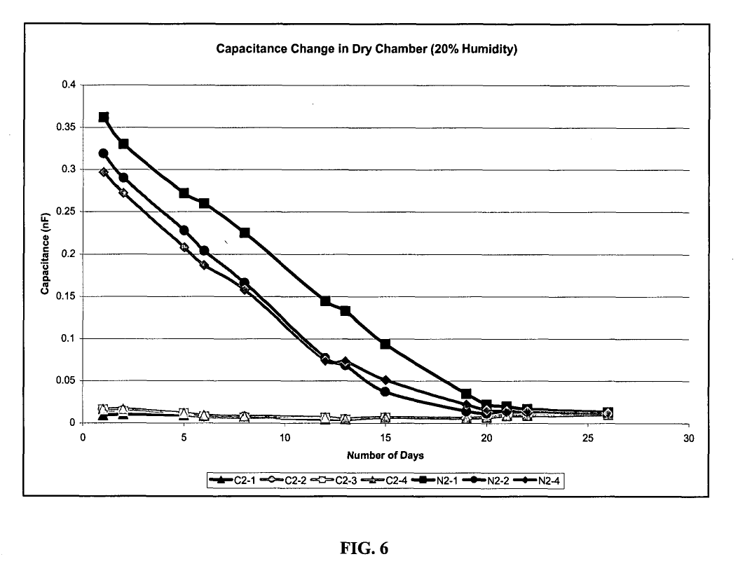

FIG. 6 is a plot of the capacitance change of N2 and C2 streaked chips in a dry environment over a period of 26 days. Humidity was kept constant at 20%. The data in TABLE 1 gives the results of some preliminary work. For this experiment, six ζ-grip microarray chips were streaked. The first two (NiCl₂-1 and NiCl₂-2) were streaked with a 0.1% solution of NiCl₂. The next two chips (C2-1 and C2-2) were streaked with a 1:10 dilution of γ-Human IgG-AP in ddH₂O. And the final two chips (N2-1 and N2-2) were streaked with a solution of the Ni-ab complex. After drying and measuring, the results in TABLE 1 were obtained. As can be seen, the NiCl₂ solution does not conduct. This is due to the hydrophobicity of the solution, as the ζ-Grip™ microarray chip will only bind hydrophilic substances. The C2 chips do not conduct as well compared to N2. The signal for N2 is about four to eight times greater than C2 in terms of current and about ten times greater in terms of capacitance.

Other experimental results further illustrate the feasibility of this detection system. FIG. 2 gives a plot of the capacitance of nickel anti-human IgG (AP) conjugates as a function of the amount of antibody for four different percentages of nickel (0%, 0.1%, 0.5%, and 1%), where the experiment was conducted at room temperature (˜25° C.) and room humidity (˜50% to 60%). FIG. 3 utilizes the same samples, but plots the current of nickel anti-human IgG (AP) conjugates, rather than capacitance.

As expected, both plots give similar trends, as current and capacitance are directly proportional. There are also apparent trends within these plots—as the amount γ-Human IgG-AP (with the same level of nickel) increases, the capacitance/current signal decreases. Also, as the amount of nickel increases, the capacitance/current signal also increases (at the same amount of antibody). This is expected, as the more nickel is apparent, the more “closed” the circuit is, allowing for more of the current to pass to the detection system.

For FIGS. 2 and 3, there is an apparent peak at ˜5 μl/ml of antibody. There is no signal at 0 μl/ml antibody, as charged nickel does not bind to the ζ-Grip™ chip. Nickel will only be apparent if the γ-Human IgG-AP antibody is bound to it. Still, the experiment was repeated for the range of 0 μl/ml to 20 μl/ml antibody, with samples streaked in increments of 2 μl/ml, so as to find the actual peak location. FIGS. 4 and 5 illustrate the plots of these results. FIG. 4 is a plot of the capacitance of nickel anti-human IgG (AP) conjugates as a function of the amount of antibody for four different percentages of nickel (0%, 0.1%, 0.5%, and 1%), where FIG. 5 plots current for the same sample rather than capacitance. Each sample is streaked three times, where each streak is measured three times. Plotted data is the average of these results.

Again, both plots give similar trends, as current and capacitance are directly proportional. Also, as the amount of nickel increases, the capacitance/current signal also increases (at the same amount of antibody). This is consistent with FIGS. 2 and 3. As for the magnitude of capacitance and current at the same level of nickel, the data fluctuates too much to come to a definite value for the peak level.

Our earlier work had demonstrated that a Ni-ab complex can be used to distinguish diseased serum from control using the standard autoantigen assay described above. Four chips were used, each with a different condition—chip #1 had no antigen streaked across it; chip #2 was not incubated in any serum; chip #3 utilized a pool of 8 SLE patients' sera; and chip #4 utilized a pool of 8 age/sexed match control patients. Chip #'s 1 and 2 showed no signal or color change, whereas chip #3 and chip #4 resulted in a voltage of 2.640 and 172.1. Chip #1 had no antigen streaked across it. Then, because the chip was blocked prior to incubating in serum, the serum had nothing to bind to, and thus the ab of the Ni-ab complex had nothing to bind to either. Therefore, no nickel was available to cause a signal. For chip #2, no serum was applied, and so the ab of the Ni-ab complex could not bind to the chip. Again, no nickel was available on the chip to cause a signal. As for chip #'s 3 and 4, the signals are significantly different. The former has a much lower signal, indicating that the conductivity is high, thus resulting in a low voltage. This follows from the general equation:

$$V = \frac{I}{g} \qquad ( 1 )$$

where, V is the voltage (mV)

I is the current (A)

G is the conductance (mho)

The inverse is true for chip #4. Only the patient serum (and not the control serum) significantly binds to the antigen. Therefore, after incubating in the Ni-ab solution, only chip # 3 would be predicted to show conductivity. This is apparent in its low voltage readout. For chip #4, there is a small amount of conductivity, resulting in high voltage readout. The difference between these two chips is significant, allowing for a clear distinction between diseased and healthy patient serum.

To verify results, chips were incubated in BNCIP reagent (to develop a visible colormetric reaction). The line appears only on chip #'s 3 and 4 (data not shown), indicating that the SSA/RO antigen, human disease related antibodies, and Ni-ab are all present.

Another experiment has shown that protein deterioration can be monitored using this system. Several C2 and N2 chips were stored in a dry chamber with anhydrous calcium sulfate and a humidity monitor. The capacitance was measured over a period of 26 days, with results illustrated in FIG. 6. This plot shows that protein deteriorates over a period of approximately 4-wks in a room temperature, low humidity environment. In addition, it further illustrates that charged nickel is a preferred metal, as no signal was detected for C2-1, C2-2, C2-3 and C2-4.

FIG. 6 is also in conjunction with the fact that this system can be modeled as a capacitor in parallel with an increasing value resistor. An experiment has been run, in which different testing strategies were used to determine an electrical model. The digital multimeter was used to measure current, resistance, and capacitance, where these values were then observed for stability. Since current fluctuates a lot, it is suspected that this “bioswitch” is not a linear resistive element. This speculation can be verified by observing the measurement of resistance. The same pattern is observed and therefore the bioswitch cannot be modeled as a pure resistor. The capacitance measurements indicate that it is charging and discharge in a certain range. Therefore, we can conclude that the bioswitch is a capacitor in parallel with a resistor. The resistor is present as protein itself degrades over time.

Although the present invention has been described in terms of certain preferred embodiments, other embodiments of the invention including variations in dimensions, configuration and materials will be apparent to those of skill in the art in view of the disclosure herein. In addition, all features discussed in connection with any one embodiment herein can be readily adapted for use in other embodiments herein. The use of different terms or reference numerals for similar features in different embodiments does not imply differences other than those which may be expressly set forth. Accordingly, the present invention is intended to be described solely by reference to the appended claims, and not limited to the preferred embodiments disclosed herein.

What is claimed is:

1. A method for detecting a disease or autoimmune state in a mammal, comprising:
immobilizing a disease or autoimmune antigen on a substrate to form a spot;
contacting said immobilized antigen with a sample from said mammal under conditions such that any antibodies from said mammal specific for said antigen bind thereto;
contacting said antibodies from said mammal bond to said antigen with a metal-antibody conjugate, wherein the antibody of the metal-antibody conjugate is specific for IgG of the species of mammal being tested;
applying a voltage between electrodes which are separated by said spot; and
measuring a current across said spot, wherein conduction of a current is indicative of the disease or autoimmune state.

2. A method for detecting the presence of an antigen, comprising:
immobilizing an antibody specific for said antigen on a substrate to form a spot;
contacting said immobilized antibody with a sample under conditions such that any antigen present in the sample binds to said immobilized antibody;
contacting said immobilized antibody-bound antigen with a metal-antibody conjugate, wherein the antibody of the metal-antibody conjugate is specific for said antigen;
applying a voltage between electrodes which are separated by said spot; and
measuring a current across said spot, wherein conduction of a current is indicative of the presence of said antigen.

3. A method for monitoring protein degradation, comprising:
a) immobilizing a metal-protein conjugate on a substrate to form a spot;
b) applying a voltage between electrodes which are separated by said spot;
c) measuring a current across said spot; and
d) repeating steps b-c over time to monitor a change in the current, which change is related to protein degradation.
